(19) United States
(12) Patent Application Publication
(10) Pub. No.: US 2003/0187075 A1
(43) Pub. Date: Oct. 2, 2003

(54) Sponge antitumor compounds

(76) Inventors: Tanaka Keith Junichi, Okinawa (JP); Higa Tatsuo, Okinawa (JP)

Correspondence Address: BANNER & WITCOFF, LTD., 28 STATE STREET, 28th FLOOR, BOSTON, MA, 02109-9601, US

(21) Appl. No.: 10/239,116

(22) PCT Filed: Mar. 20, 2001
(86) PCT No.: PCT/GB01/01213

(30) Foreign Application Priority Data
Mar. 20, 2000 (GB) 00067413

(51) Int. Cl. A61K 31/13; A61K 31/11

(52) U.S. Cl. 514/641; 564/271; 568/445; 514/693

(57) ABSTRACT

New antitumor compounds isolated from a sponge are of the a formulae (1), (2), (3) and (4).

The present invention relates to antitumor compounds from a sponge.

### BACKGROUND OF THE INVENTION

We recently reported the isolation and structure elucidation of two new sesquiterpene carbonimidic dichlorides (8,10) from the nudibranch Reticulidia fungia.¹

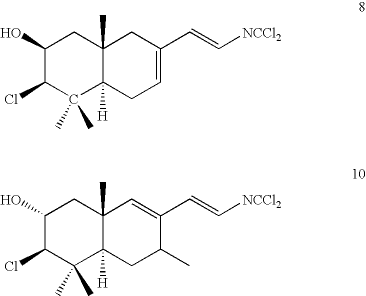

Inasmuch as related compounds have earlier been described only from a few species of sponges,²⁻⁶ it was evident that the nudibranch constitutes originated from a sponge. However, we were unable to locate a plausible species in the vicinity of the nudibranch collection site on Irabu Island, Okinawa.

### SUMMARY OF THE INVENTION

We examined cytotoxic constituents of a sponge collected from a coral reef off Iriomote Island, located 150 km west of Irabu, and later identified as Stylotella aurantium, the same species that yielded carbonimidic dichlorides in Australia.⁶ Our sample also gave sesquiterpene carbonimidic dichlorides, including five new congeners (1-5), which were responsible for the cytotoxicity of the lipophilic extract of the sponge.

Thus we provide five new sesquiterpenes (1-5) having a carbonimidic dichloride or an aldehyde function which have been isolated, and which occur together with seven known related compounds (6-12), from the sponge Stylotella aurantium. The structures of the new compounds were elucidated from spectral data. The absolute stereochemistry of the previously reported reticulidin A (10) was determined. Four of the new compounds showed cytotoxicity with a range of IC₅₀ values of 0.1-1 μg/mL against several tumour cell lines.

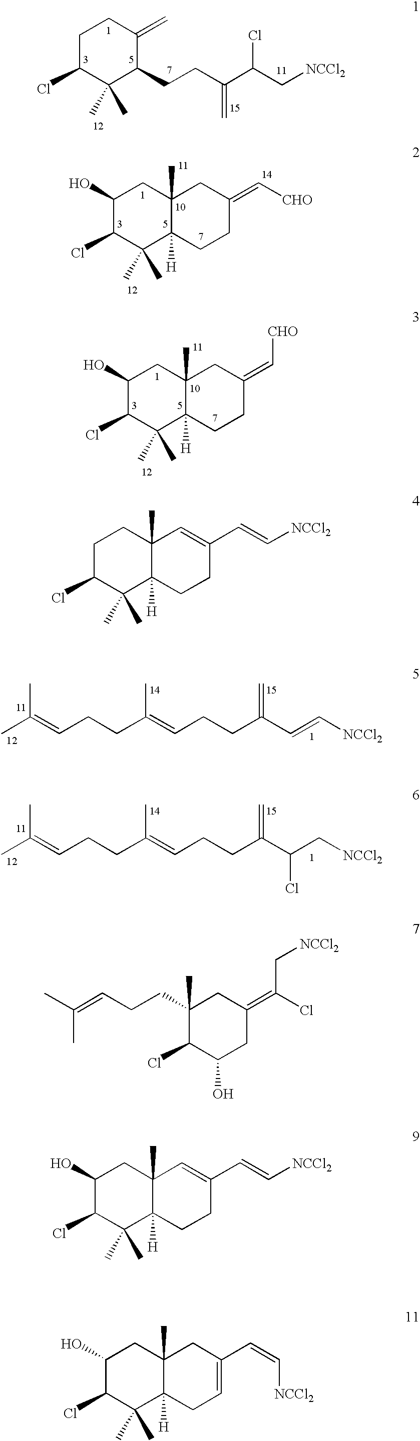

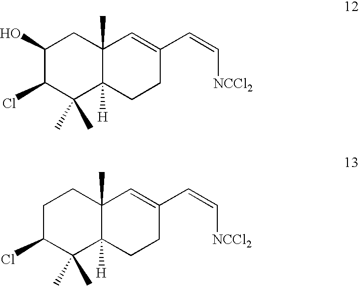

The sponge (OP-98-30) was identified to be Stylotella aurantium Kelly-Borges & Bergquist, 1988 (Porifera, Demospongiae, Halichondrida, Axinellidae) by Dr. J. N. A. Hooper, Queensland Museum. A voucher specimen (QM G317008) is deposited at Queensland Museum, South Brisbane, Australia.

### ANTITUMORAL ACTIVITY

| | IC₅₀ (μg/ml) | | | |
|---|---|---|---|---|
| Com- | P388 | A549 | HT29 | MEL28 |
| pound | Leukaemia | (Human Lung) | (Human Colon) | (Melanoma) |
| 1 | 1 | 0.1 | 0.1 | 0.1 |
| 2 | 1 | 1 | 1 | 1 |
| 3 | 1 | 1 | 1 | 1 |
| 4 | 1 | 1 | 1 | 1 |

### PREFERRED EMBODIMENTS

In view of the in vitro activity, the compounds of this invention, compounds (1) to (4), are expected to be useful in the treatment of cancer.

Thus, the present invention provides a method of treating any mammal, notably a human, affected by cancer which comprises administering to the affected individual a therapeutically effective amount of a compound of the invention, or a pharmaceutical composition thereof.

The present invention also relates to pharmaceutical preparations, which contain as an active ingredient a compound of the invention, as well as the processes for their preparation.

Examples of pharmaceutical compositions include any solid (tablets, pills, capsules, granules, etc.) or liquid (solutions, suspensions or emulsions) with suitable composition or oral, topical or parenteral administration, and they may contain the pure compound or in combination with any carrier or other pharmacologically active compounds. These compositions may need to be sterile when administered parenterally.

Administration of the compounds or compositions of the present invention may be by any suitable method, such as intravenous infusion, oral preparations, intraperitoneal and intravenous administration. We prefer that infusion times of up to 24 hours are used, more preferably 2 to 12 hours, with 2 to 6 hours most preferred. Short infusion times which allow treatment to be carried out without an overnight stay in hospital are especially desirable. However, infusion may be 12 to 24 hours or even longer if required. Infusion may be carried out at suitable intervals of say 2 to 4 weeks. Pharmaceutical compositions containing compounds of the invention may be delivered by liposome or nanosphere encapsulation, in sustained release formulations or by other standard delivery means.

The correct dosage of the compounds will vary according to the particular formulation, the mode of application, and the particular situs, host and tumour being treated. Other factors like age, body weight, sex, diet, time of administration, rate of excretion, condition of host, drug combinations, reaction sensitivities and severity of the disease shall be taken into account. Administration can be carried out continuously or periodically within the maximum tolerated dose.

The compounds and compositions of this invention may be used with other drugs to provide a combination therapy. The other drugs may form part of the same composition, or be provided as a separate composition for administration at the same time or a different time. The identity of the other drug is not particularly limited, and suitable candidates include:

a) drugs with antimitotic effects, especially those which target cytoskeletal elements, including microtubule modulators such as taxane drugs (such as taxol, paclitaxel, taxotere, docetaxel), podophylotoxins or vinca alkaloids (vincristine, vinblastine);

b) antimetabolite drugs such as 5-fluorouracil, cytarabine, gemcitabine, purine analogues such as pentostatin, methotrexate);

c) alkylating agents such as nitrogen mustards (such as cyclophosphamide or ifosphamide);

d) drugs which target DNA such as the antracycline drugs adriamycin, doxorubicin, pharnorubicin or epirubicin;

e) drugs which target topoisomerases such as etoposide;

f) hormones and hormone agonists or antagonists such as estrogens, antiestrogens (tamoxifen and related compounds) and androgens, flutamide, leuprorelin, goserelin, cyprotrone or octreotide;

g) drugs which target signal transduction in tumour cells including antibody derivatives such as herceptin;

h) alkylating drugs such as platinum drugs (cis-platin, carbonplatin, oxaliplatin, paraplatin) or nitrosoureas;

i) drugs potentially affecting metastasis of tumours such as matrix metalloproteinase inhibitors;

j) gene therapy and antisense agents;

k) antibody therapeutics; and

l) other bioactive compounds of marine origin, notably the ecteinascidins such as Et-743 or the didemnins such as aplidine.

The present invention also extends to the compounds of the invention for use in a method of treatment, and to the use of the compounds in the preparation of a composition for treatment of cancer.

### EXAMPLES OF THE INVENTION

A sample (90 g, wet wt) of S. aurantium Kelly-Borges and Bergquist (family Axinellidae) was extracted with acetone, and the concentrated extract was partitioned between ethyl acetate and water. The EtOAc extract (0.65 g) was separated on Si gel followed by preparative TLC and/or HPLC to afford sesquiterpenes 1-12 in yields ranging from 1.5 to 12.0 mg.

General Experimental-Procedures. IR spectra were measured on a JASCO FT/IR 300 and UV spectra on a UVIDEC 610 spectrophotometer. NMR spectra were recorded on a JEOL A500 instrument at 500 Mhz (¹H) and 125 Mhz (¹³C). LREIMS and HREIMS were obtained using a Hitachi M-2500 mass spectrometer. Optical rotation was taken on a JASCO DIP-1000 polarimeter.

Animal Material. A specimen (90 g, wet wt) of the title sponge was collected by hand using scuba at −15 m in Iriomote Island, Okinawa in May 1998. A voucher specimen (QM G317008) is deposited at Queensland Museum, Brisbane, Australia, and the sample was identified by Dr. John N. A. Hooper, Natural Environment Program, Queensland Museum, South Brisbane, Queensland, Australia

Extraction and Isolation. The sponge sample was kept frozen until extraction. The whole animal was extracted three times with Me₂CO (500 mL). The combined extracts were concentrated in vacuo, and the residue partitioned between EtAOc and H₂O to obtain a lipophilic extract (0.70 g). Most of the extract (0.65 g) was separated on a Si gel column by eluting stepwise with heptane, heptane-CH₂Cl₂, CH₂Cl₂, CH₂Cl₂-EtOAc, EtOAc, EtOAc-MeOH, and MeOH to give nine fractions. The first fraction (12.7 mg) was further separated by preparative TLC (SiO₂, heptane-CH₂Cl₂, 10:1) followed by HPLC (RP₁₃, MeOH-H₂O, 15:1) to give compounds 5 (2.0 mg) and 6 (1.9 mg). The second fraction (39.8 mg) was separated by HPLC (SiO₂, heptane-CH₂Cl₂, 9:1) to give compounds 1 (6.1 mg), 4 (1.6 mg), and 6 (8.0 mg). The fourth fraction (64.0 mg) was separated by HPLC (SiO₂, heptane-CH₂Cl₂, 3:1) to give 11 (1.5 mg) and 12 (1.7 mg). The fifth fraction (88.7 mg) was repeatedly separated by preparative TLC (SiO₂, first: heptane-EtOAc, 9:1; second: heptane-CH₂Cl₂, 3:2; third: CH₂Cl₂) to give 2 (1.6 mg), 3 (1.6 mg), 8 (9.1 mg), and 9 (5.8 mg). The sixth fraction (184.5 mg) was separated on a Si gel column (heptane-CH₂Cl₂-EtOAc) followed by preparative TLC (heptane-CH₂Cl₂, 3:2) to give 7 (12.0 mg) and 10 (4.6 mg).

Compound 1: colourless oil; [α]²⁵_{D}+4.5° (c 0.20, CHCl₃); UV (MeOH) λₘₐₓ (log ε) 205 (3.6 nm; IR (neat) νₘₐₓ 1655, 877 cm⁻¹; ¹H NMR (CDCl₃) δ0.84 (3H, s, H₃-13), 1.16 (3H, s, H₃-12), 1.74 (1H, m, H-7a), 1.77 (1H, m, H-5), 1.81 (1H, m, H-7b), 1.86 (1H, m, H-2β), 1.91 (1H, m, H-8a), 2.04 (1H, dt, J=4, 13 Hz, H-1α), 2.12 (1H, dq, J=13.4 Hz, H-2α), 2.38 (1H, m, H-8b), 2.41 (1H, m, H-1β), 3.84 (2H, d, J=7 Hz, H-11), 3.90 (1H, dd, J=4, 11 Hz, H-3) 4.60 (1H, t, J=7 Hz, H-10), 4.66 (1H, s, H-14a), 4.95 (1H, s, H-14b), 5.07 (1H, s, H-15a), 5.19 (1H, s, H-15b); ¹³C NMR (CDCl₃) δ15.9 q (C-13), 24.3 t (C-7), 27.2 q (C-12), 30.7 t (C-8), 34.5 t (C-2), 35.4 t (C-1)-4) 52.4 d (C-5), 59.3 t (C-11), 62.2 d (C-10), 70.9 d (C-3), 109.2 t (C-14), 114.4 t (C-15), 127.0 s (C-16), 145.6 s (C-6), 146.3 s (C-9); ESIMS m/z 369 ([M⁺], 62 rel %); EIMS m/z 334 ([M−Cl]⁺, 100), 336 (98), 338 (32), 298 (65), 300 (40), 302 (7), 262 (30), 264 (12 rel %): HREIMS m/z 334.0870 (calcd for C₁₆H₂₅³⁵Cl₃N, 334.0894).

Compound 2: colorless oil, [α]²⁵_{D}+16° (c 0.13, CHCl₃), UV (MeOH)λₘₐₓ(log ε) 235 (3.3)nm; IR (neat)νₘₐₓ 3458, 1714, 1668 cm⁻; ¹H NMR (CDCl₃) δ1.11 (6H, S, H₃-12,13), 1.14 (3H, s, H₃-11), 1.98 (1H, dd, J=3, 12 Hz, H-5), 1.52 (1H, br d, J=14 Hz, H-1α), 1.65 (1H, dq, J=4, 13 Hz H-6β), 1.98 (1H, m, H-6α), 2.02 (2H, m, H₂-9), 2.05 (1H, m, H-7α), 2.06 (1H, dd, J=3, 14 Hz, H-1β), 2.35 (1H, br s, OH), 3.50 (1H, br d, J=14 Hz, H-7β), 3.94 (1H, d, J=3 Hz, H-3), 4.18 (1H, q, J=3 Hz, H-2), 5.78 (1H, d, J=8 Hz, H-14), 10.02 (1H, d, J=8 Hz, H-15); ¹³C NMR (CDCl₃) δ17.8 (C-13) 20.8 q (C-11), 23.9 t (C-6), 29.5 t (C-7), 30.4 q (C-12), 36.7 s (C-10), 39.5 s (C-4), 45.1 t (C-1), 54.1 d (C-5), 55.8 t (C-9), 71.9 d (C-2), 76.3 d (C-3), 127.5 d (C-14), 163.5 s (C-8), 190.1 d (C-15) EIMS m/z 270 (M⁺, 100), 272 (33), 255 (18), 226 (48), 217 (74 rel %); HREIMS m/z 270.1363 (calcd for C₁₆H₂₃ClCO₂, 270, 1384).

Compound 3: colorless oil, [α]^{2G}_{D}−28° (c 0.13, CHCls), UV(MeOH) λₘₐₓ (log ε) 24-0 (3.9), 285 (3.1) nm; IR (neat) νₘₐₓ 3467, 1716, 1651 cm⁻¹; ¹H NMR (CDCL₂) δ1.09 (3H, s, H₃-12), 1.10 (3H, s, Hₛ-13), 1.16 (3H, s, Hₛ-11), 1.38 (1H, dd, J=2.5, 13.0 Hz, H-5), 1.58 (1H, m, H-1α), 1.67 (1H, dq, J=4.0, 13.0 Hz, H-6β), 1.80 (1H, d, J=13.0 Hz, H-9α), 1.96 (1H, m, H6α), 2.09 (1H; dd, J=2.5, 14.5 Hz, H-1β), 2.26 (1H, dt, J=6.0, 13.0 Hz, H-7α), 2.36 (1H, s, OH), 2.49 (1H, br d, J=13.0 Hz, H-7β), 3.02 (1H, br d, J=13.0 Hz, H-9β), 3.94 (1H, d, J=3.0 Hz, H-3), 4.19 (1H, br s, H-2), 5.95 (1H, d, J=8.1 Hz, H-14), 9.95 (1H, d, J=8.1 Hz, H-15); ^{1B}C NMR (CDCL₃) δ17.8 q (C-13), 20.9 q (C-11), 24.2 t (C-6), 30.3 q (C-12), 36.5 a (C-10), 37.9 t (C-7), 39.5 a (C-4), 45.1 t (C-1), 47.2 t (C-9), 54.2 d (C-5), 71.9 d (C-2), 76.3 d (C-3), 127.7 d (C-14), 163.7 s (C-8), 190.5 d (C-15); EIMS m/z 270 (M⁺, 98), 272 (34), 255 (42), 226 (70),217 (100 rel %); HREIMS m/z 270.1400 (callcd for C₁₅H₂₃^{3G}C10₂,270.1384).

Compound 4: colorless oil, [α]²⁵_{D}+40°(c 0.13, CHCl₃), UV (MeOH) λₘₐₓ (log ε) 288 (4.2) nm; IR (neat) νₘₐₓ 1640; 900 cm⁻¹; ¹HNMR (CDCl₃) δ0.94 (3H, s, H₃-13), 1.03 (3H, s, H₃-11), 1.10 (3H, s, H₃-12), 1.20 (1H, dd, J=2, 13 Hz, H-5), 1.38 (1H, dt, J=4, 13 Hz, H-1a), 1.57 (1H, t, J=3 Hz, H-6β), 1.59 (1H, t, J=3 Hz, H-1β), 1.89 (1H, br dd, J=7, 13 Hz, H-6a), 2.01 (1H, m, H-2α), 2.07 (1H, m, H-2β), 2.21 (1H, m, H-7α), 2.36 (1H, dd, J=6, 17 Hz, H-7β), 3.75 (1H, dd, J=5, 12 Hz, H-3), 5.59 (1H, s, H-9), 6.51 (1H, d, J=13 Hz, H-14), 6.70 (1H, d, J=13 Hz, H-15); ¹³C NMR (CDCl₂) δ16.6 q (C-13), 19.2 t (C-6), 20.8 q (C-11), 26.2 t (C-7), 28.9 q (C-12), 29.8 t (C-2), 36.0 s (C-10), 38.8 t (C-1), 40.0 s (C4), 51.3 d (C-5), 72.4 d (C-3), 124.0 s (C-16), 128.9 d (C-15), 130.6 s (C-8), 137.7 d (c-14), 146.4 d (C-9); EIMS m/z 333 (M⁺, 52), 335 (52), 337 (17), 339 (1), 298 (100, 300 (65), 302 (12), 262 (75), 264 (26), 226 (36 rel %); HREIMS m/z 333.0823 (calcd for C₁₆H₂₂²⁸ClₛN, 333.0816).

Compound 5: colorless oil, ¹H NMR (CDCl₃) δ1.61 (6H, s, H₃-13, 14), 1.68, s, H₃-12), 1.99 (2H, t, J=7 Hz, H-8), 2.07 (2H, q, J=7, 13 Hz, H-9), 2.23 (2H, m, H-5), 2.28 (2H, m, H-4), 5.09 (1H, br t, J=7 Hz, H-10), 5.16 (1H, tq J=7.2 Hz, H-6), 5.19 (1H, br s, H-15a), 5.23 (1H, br s, H-15b), 6.62 (1H, d, J-13 Hz, H-2), 6.87 (1H, d, J=13 Hz, H-1); ¹³C NMR (C-5), 26.7 t (C-9), 32.1 t (C-4), 39.7 t (C-8), 120.3 t (C-15), 123.4 d (C-6), 124.3 d (C-10), 125.2 s (C-16), 130.8 d (C-1), 131.4 s (C-11), 135.9 s (C-7, 137.0 d (C-2), 143.9 s (C-3).

(R)-MTPA Ester of 10. A mixture of 0.035 mg of reticulidin A (10), 0.31 mg of DCC, 0.35 mg of (R)-MTPA, and 0.12 mg of DMAP in 0.15 mL of CH₂Cl₂ was kept standing at room temperature for 3 h. After removal of the solvent, the residue was separted by preparative-TLC (SiO₂, hexane-EtOAc, 4:1) to afford 0.30 mg of (R)-MTPA ester: ¹H NMR (CDCl₃) δ1.034 (3H, s, H₃-13), 1.156 (6H, s, H₂-11, 12), 2.025 (1H, dd, J=4, 12 Hz, H-1β), 3.741 (1H, d, J=11 Hz, H-3), 5.387 (1H, dt, J=5, 11 Hz, H-2), 5.562 (1H, s, H-9), 6.486 (1H, d, J=13 Hz, H-14), 6.712 (1H, d, J=13 Hz, H-15).

(S)-MTPA Ester of 10. The ester was similarly prepared as above using (S)-MTPA: ¹H NMR (CDCl₃) δ1.028 (3H, s, H₃-13), 1.148 (3H, s, H₃-12), 1.172 (3H, s, H₃-11) 2.112 (1H, dd, J=4, 12 Hz, H-1β, 3.720 (1H, d, J=11 Hz, H-3), 5.417 (1H, dt, J=5, 11 Hz, H-2), 5.610 (1H, s, H-9), 6.507 (1H, d, J=13 Hz, H-14), 6.729 (1H, d, J=13 Hz, H-15.

Treatment of Reticulidin B (8) with an Acid. A mixture of 1.0 mg of reticulidin B (8) and a catalytic amount of p-toluenesulfonic acid monohydrate in 0.5 mL of 30% aqueous THF was kept standing at room temperatue for 12 h. The reaction mixture showed only one spot on TLC (SiO₂, heptane-EtOAc, 3:2), and unreacted 8 (0.8 mg) was recovered.

Compound 1, [α]_{D}+4.5° (c 0.20, CHCl₃) was isolated as a colourless oil. The molecular formula C₁₆H₂₃NCl₄ was determined by observing a molecular ion at m/z 369 in ESIMS and by HREIMS at m/z 334.0870 ([M−Cl]⁺). The presence of a carbonimidic dichloride functional group was inferred from a carbon signal at δ127.0 s and also by IR absorption at 1655 cm⁻¹, as reported earlier.²⁻⁶ The ¹H and exomethylenes [δ4.66 s, 4.95 s, 5.07 s, 5.19 s; δ109.2 t (C-14), 114.4 t (C-15), 145.6 s (C-6), 146.3 s (C-9)], two chlorine-bearing methines [δ3.90 dd, 4.60 t; δ62.2 d (C-10), 70.9 d (C-3)], a methylene bearing a nitrogen [δ3.84 d; δ59.3 t (C-11)], and two methyls [δ0.84s, 1.16s; δ15.9 q (C-12), 27.2 q (C-13)]. These data, together with the unsaturation requirement, suggested 1 to be monocylic. Connectivity was made by interpreting 2D NMR (COSY, HMQX, HMBC) spectra. The presence of a six-membered ring was shown by COSY (H-1αβH-2αβ, H-2αβ, H-2αβ/H-3, H-1α/H-5, H-1α/H-14a, H-1β/H-14b, and H-5/H-14ab) and HMBC data (H-1αβ/C-2,-3,-5,-6,-14, H-2αβ/C-1,-3,-4,-6, H-3/C-4,-12,-13, H-5/C-3,-4,-6,-8,-14, and Hₐ-12,-13/C-3,-4,-5). Connectivity between the ring and the terminal carbonimidic dichloride was also made by COSY (H-5/H-7ab,-8ab; H-7ab/H-8ab, H-8ab/H-15ab, H-10/H-11,-15b) and HMBC (H-10/C-15, H₂-11/C-16) cross-peaks. Relative stereochemistry in the ring was elucidated as shown by observing positive NOEs (H-3/H-5, H-3/Hₐ-12, H-7/H₃-13). The stereochemistry at C-10 remains to be solved.

Compound 2, [α]_{D}+16° (c 0.13, CHCl₃), was obtained as a colourless glass. EIMS of 2 showed a molecular ion at m/z 270. High-resolution measurement of this peak gave a molecular formula C₁₆H₂₅ClO₂. The formula indicated four degrees of unsaturation. The NMR spectra displayed signals for three methyl singlets [δ1.11 (Hₐ-12), 1.11 (H₃-13), 1.14 (H₃-11); δ17.8 q, 23.9 t, 30.4 q], α,β-unsaturated aldehyde [δ10.02 d; δ127.5 d (C-14), 163.5 s (C-8), 190.1 d (C-15)], and two methines bearing a chlorine and/or a hydroxyl [δ3.93 d, 4.18 q; δ71.9 d (C-2), 76.3 d (C-3)]. The IR absorption band at 1666 cm⁻¹ also indicated the presence of the α,β-unsaturated aldehyde. Comparison of these and 2D NMR data with those of reticulidin B (8) suggested that 2 consisted of a bicyclic portion similar to reticulidin B and an enal moiety instead of a carbonimidic dichloride as in 8. The hydroxyl group was located at C-2 by a deuterium-induced shift experiment (Δδ-0.115), as before.¹ The double-bond geometry of the enal was assigned as E by positive NOEs between H-9 and H-14 and also between H-7β and H-15. Compound 2 had the same relative stereochemistry as 8 as confirmed by NOE observation (H-2/H-3, H-3/H-5). Because the aldehyde could be derived by hydrolysis of 8, it was suspected that 2 might be an artifact formed during the isolation procedure. However, when 8 was treated with p-TsOH in aqueous THF (room temperature, 12 h), 8 was recovered with no signs of reaction, suggesting that 2 is, indeed, a natural product.

Compound 3, [α]_{D -}28° (c 0.13, CHCl₃), had the same molecular formula, C₁₆H₂₃ClO₂, as 2 as determined by HREIMS. The ¹³C NMR spectrum of 3 was almost identical to that of 2, except for the signals for C-7 (Δδ8.4) and C-9 (Δδ-8.6). in the ¹H NMR spectrum major differences between 3 and 2 were noted for the chemical shifts for H-7 (Δδ0.21, -1.01), H-9 (-0.22, 1.00), H-14 (0.17), and H-15 (-0.07), suggesting a configurational difference around the double bond. Observation of positive NOEs (H-7β/H-14, H-9β/H-15) revealed the Z-configuration of the double bond. Relative stereochemistry on the bicyclic portion was the same as that of 2, as confirmed by NOE measurements. The position of the hydroxyl group on C-2 was also confirmed by a deuterium-induced shift (Δδ-0.115).

Compound 4, [α]_{D}+40° (c 0.13, CHCl₃), had the molecular formula C₁₆H₂₂NCl₃ (HREIMS, Δ+0.7 mmu). It contained a carbonimidic dichloride (1640 cm⁻¹; δ124.0 s), two double bonds [δ5.59 s (H-9), 6.51 d (H-14), 6.70 d (H-15); δ128.9 d, 130.6 s, 137.7 d, 146.4 d], a chlorine-bearing methine (δ3.75 dd; δ72.4 d), and three methyls [δ0.94 s (H-13), 1.03 s (H-11), 1.10 s (H-12); δ16.6 q, 20.8 q, 28.9 q]. The structure of 4 was secured by COSY, HMQC, and HMBC data and also be comparison of these data with those of 9 and 13.4 The relative stereochemistry is based on the NOESY cross-peaks (H-3/H-2α, H-3/H-5, H-3/H₃-12, H-5/H₃-12, H₃-11/H₃-13) and on the similarity of NMR data to shoe of 13.⁴ Finally, the coupling constant J_{14,15} (13.0 Hz) and NOEs between H-9 and H-14 and also between H-7β and H-15 were indicative of E geometry of the disubstituted double bond.

Because compound 5 decomposed during storage in an NMR tube, we failed to record mass spectral data. A plausible formula, C₁₆H₂₃NCl₂, could be deduced from NMR data. The ¹H NMR spectrum of 5 was composed of signals for two exomethylene protons [δ5.19 and 5.23 s (H-15)], four vinyl protons [δ5.09 t (H-10), 5.16 t (H-6), 6.62 d (H-2), and 6.87 d (H-1)], four methylenes [δ1.99 t (H-8), 2.07 q (H-9), 2.23 q (H-5), 2.28 t (H-4)], and three vinyl methyls [δ1.61 s (H-13), 1.61 s (H-14), 1.69 s (H-12)]. In addition to a characteristic signal at δ124.2 s for NCCl₂, the ¹³C NMR data, together with an HMQC experiment, confirmed the presence of the above functionalities. Comparison of these data with those of 6 suggested that 5 has a trans double bond (J=13.1 Hz) in the place of the methylene (C-1) and chloromethine (C-2) in 6.

The absolute stereochemistry of reticulidin A (10) was determined by modified Mosher's method.⁷ When NMR spectra were recorded with MTPA derivatives of 10, positive values (Δδ_{s-R}) were observed for H-1β(+0.087), H₃-11 (+0.016), and H-9 (+0.048), while negative values were detected for H-3 (−0.021), H₃-12 (−0.008), and H₃-13 (−0.006). Therefore, the absolute configuration of 10 was determined as (2R, 3R, 5S, 10S). Reticulidin B (8) did not form MTPA esters when treated with MTPA, DCC, and DMAP, presumably due to steric hindrance of the hydroxyl group. However, from the result with 10 and their close structural relationship, it could be concluded that the absolute stereochemistries of 8-12 and 2 and 3 are as depicted on the structures.

The aldehydes 2 and 3 could possible be catabolic products of corresponding carbonimidic dichlorides. However, the possibility of their role as biosynthetic precursors could not be ruled out. At this point we have no conclusive evidence to determine their biosynthetic relationship.

### REFERENCES AND NOTES

(1) Tanaka, J.; Higa, T. J. Nat. Prod. 1999, 62, 1339-1340.

(2) Wratten, S. J.; Faulkner, D. J. J. Am. Chem. Soc. 1977, 99, 7367-7368.

(3) Wratten, S. J.; Faulkner, D. J.; Van Engen, D.; Clardy, J. Tetrahedron Lett. 1978, 1391-1394.

(4) Wratten, S. J.; Faulkner, D. J. Tetrahedron Lett. 1978, 1395-1396.

(5) Hirota, H.; Okino, T.; Yoshimura, E.; Fusetani, N. Tetrahedron 1998, 54, 13971-13980.

(6) Simpson, J. S.; Raniga, P.; Garson, M. J. Tetrahedron Lett. 1997, 38, 7947-7950.

(7) Ohtani, I.; Kusumi, T.; Kashman, Y.; Kakisawa, H. J. Am. Chem. Soc. 1991, 113, 4092-4096.

1. A compound selected from the group consisting of compounds (1) to (4) of the following formulae:

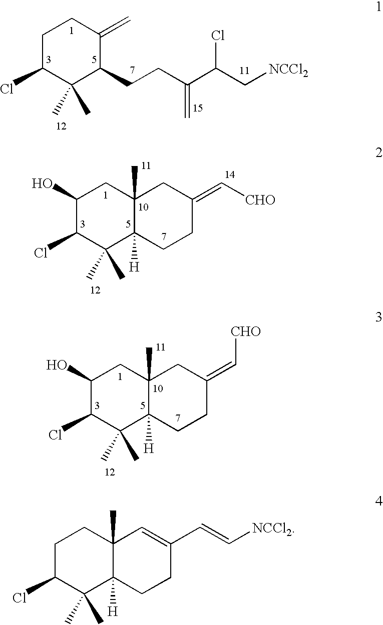

2. A pharmaceutical composition containing a compound of claim 1 together with a pharmaceutically acceptable carrier or diluent.
